(19) United States
(12) Patent Application Publication
(10) Pub. No.: US 2004/0152753 A1
(43) Pub. Date: Aug. 5, 2004

(54) Methods of use for novel sulfur containing organic nitrate compounds

(75) Inventors: David S. Garvey, Dover, MA (US); L. Gordon Letts, Dover, MA (US)

Correspondence Address: EDWARD D GRIEFF, HALE & DORR LLP, 1455 PENNSYLVANIA AVE, NW, WASHINGTON, DC, 20004, US

(73) Assignee: NitroMed, Inc., Bedford, MA

(21) Appl. No.: 10/760,672

(22) Filed: Jan. 21, 2004

Related U.S. Application Data
(63) Continuation of application No. PCT/US02/24923, filed on Aug. 7, 2002.
(60) Provisional application No. 60/311,715, filed on Aug. 10, 2001.

(51) Int. Cl. A61K 31/415

(52) U.S. Cl. 514/406

(57) ABSTRACT

The invention describes methods of use for an organic nitrate compound, or a pharmaceutically acceptable salt thereof, wherein the organic nitrate compound comprises at least one sulfur atom and/or at least one disulfide group. The invention also provides methods for treating, preventing and/or reducing inflammation, pain, and fever; for decreasing or reversing the gastrointestinal, renal and other toxicities resulting from the use of nonsteroidal antiinflammatory compounds; for treating and/or preventing gastrointestinal disorders; for treating inflammatory disease states and disorders; for treating and/or preventing ophthalmic diseases or disorders; for treating and/or improving the gastrointestinal properties of COX-2 inhibitors; for facilitating wound healing; for treating and/or preventing other disorders resulting from elevated levels of cyclooxygenase-2; for decreasing the recurrence of ulcers; for improving gastroprotective properties, anti-Helicobacter pylori properties or antacid properties of proton pump inhibitors; for treating Helicobacter pylori and viral infections; for improving gastroprotective properties of H₂ receptor antagonists; for treating and/or preventing inflammations and microbial infections, multiple sclerosis, and viral infections; for treating or preventing restenosis, autoimmune diseases, pathological conditions resulting from abnormal cell proliferation, polycystic kidney disease, inflammatory diseases or to inhibit wound contraction; for treating or preventing sexual dysfunctions in males and females, for enhancing sexual responses in males and females; for treating or preventing benign prostatic hyperplasia, hypertension, congestive heart failure, variant (Printzmetal) angina, glaucoma, neurodegenerative disorders, vasospastic diseases, cognitive disorders, urge incontinence, and overactive bladder; for reversing the state of anesthesia; for treating or preventing diseases induced by the increased metabolism of cyclic guanosine 3′,5′-monophosphate (cGMP); for treating respiratory disorders and for treating neurological conditions.

### RELATED APPLICATIONS

This application is a continuation of PCT/US02/24923 filed Aug. 7, 2002, which claims priority to U.S. Application No. 60/311,715 filed Aug. 10, 2001.

### FIELD OF THE INVENTION

The invention describes methods of use for an organic nitrate compound, or a pharmaceutically acceptable salt thereof, wherein the organic nitrate compound comprises at least one sulfur atom and/or at least one disulfide group. The invention also provides methods for treating, preventing and/or reducing inflammation, pain, and fever; for decreasing or reversing the gastrointestinal, renal and other toxicities resulting from the use of nonsteroidal antiinflammatory compounds; for treating and/or preventing gastrointestinal disorders; for treating inflammatory disease states and disorders; for treating and/or preventing ophthalmic diseases or disorders; for treating and/or improving the gastrointestinal properties of COX-2 inhibitors; for facilitating wound healing; for treating and/or preventing other disorders resulting from elevated levels of cyclooxygenase-2; for decreasing the recurrence of ulcers; for improving gastroprotective properties, anti-Helicobacter pylori properties or antacid properties of proton pump inhibitors; for treating Helicobacter pylori and viral infections; for improving gastroprotective properties of H₂ receptor antagonists; for treating and/or preventing inflammations and microbial infections, multiple sclerosis, and viral infections; for treating or preventing restenosis, autoimmune diseases, pathological conditions resulting from abnormal cell proliferation, polycystic kidney disease, inflammatory diseases or to inhibit wound contraction; for treating or preventing sexual dysfunctions in males and females, for enhancing sexual responses in males and females; for treating or preventing benign prostatic hyperplasia, hypertension, congestive heart failure, variant (Printzmetal) angina, glaucoma, neurodegenerative disorders, vasospastic diseases, cognitive disorders, urge incontinence, and overactive bladder; for reversing the state of anesthesia; for treating or preventing diseases induced by the increased metabolism of cyclic guanosine 3′,5′-monophosphate (cGMP); for treating respiratory disorders and for treating neurological conditions.

### BACKGROUND OF THE INVENTION

Endothelium-derived relaxing factor (EDRF) is a vascular relaxing factor secreted by the endothelium and is important in the control of vascular tone, blood pressure, inhibition of platelet aggregation, inhibition of platelet adhesion, inhibition of mitogenesis, inhibition of proliferation of cultured vascular smooth muscle, inhibition of leukocyte adherence and prevention of thrombosis. EDRF has been identified as nitric oxide (NO) or a closely related derivative thereof (Palmer et al, Nature, 327:524-526 (1987); Ignarro et al, Proc. Natl. Acad. Sci. USA, 84:9265-9269 (1987)).

There is a need in the art for effective methods of preventing or treating numerous diseases and disorders, particularly, inflammation, pain, gastrointestinal, restenosis, sexual dysfunctions and respiratory diseases and disorders. The invention is directed to these, as well as other, important ends.

### SUMMARY OF THE INVENTION

The invention describes methods for preventing and/or treating diseases and disorders by administering at least one organic nitrate compound, or a pharmaceutically acceptable salt thereof, wherein the organic nitrate compound comprises at least one sulfur atom and/or at least one disulfide group.

One embodiment of the invention provides methods for treating, preventing and/or reducing inflammation, pain, and fever; for decreasing and/or reversing the gastrointestinal, renal and other toxicities resulting from the use of nonsteroidal antiinflammatory compounds; for treating and/or preventing gastrointestinal disorders; for facilitating wound healing; for treating inflammatory disease states and/or disorders; and for treating and/or preventing ophthalmic diseases and/or disorders in a patient in need thereof which comprises administering to the patient a therapeutically effective amount of at least one organic nitrate compound comprising at least one sulfur atom and/or at least one disulfide group. The methods can optionally further comprise the administration of at least one nonsteroidal antiinflammatory compound (NSAID) that is optionally substituted with at least one NO and/or NO₂ group (i.e., nitrosylated and/or nitrosated). In this embodiment of the invention, the methods can involve administering the organic nitrate compounds, administering the organic nitrate compounds and NSAIDs, administering the organic nitrate compounds and nitrosated NSAIDs, administering the organic nitrate compounds and nitrosylated NSAIDs, administering the organic nitrate compounds and nitrosated and/or nitrosylated NSAIDs, or administering the organic nitrate compounds, NSAIDs and nitrosated and/or nitrosylated NSAIDs. The organic nitrate compounds and NSAIDs can be administered separately or as components of the same composition in one or more pharmaceutically acceptable carriers.

Yet another aspect of the present invention provides for treating and/or improving gastrointestinal properties of COX-2 selective inhibitors; for facilitating wound healing; for treating and/or preventing renal toxicity; and for treating and/or preventing COX-2 mediated disorders (i.e., disorders resulting from elevated levels of COX-2) in a patient in need thereof which comprises administering to the patient a therapeutically effective amount of at least one organic nitrate compound comprising at least one sulfur atom and/or at least one disulfide group. The methods can optionally further comprise the administration of at least one COX-2 selective inhibitor that is optionally substituted with at least one NO and/or NO₂ group (i.e., nitrosylated and/or nitrosated). The methods can optionally further comprise the administration of at least one therapeutic agent. In this embodiment of the invention, the methods can involve administering the organic nitrate compounds, administering the organic nitrate compounds and COX-2 selective inhibitors, administering the organic nitrate compounds and nitrosated COX-2 selective inhibitors, administering the organic nitrate compounds and nitrosylated COX-2 selective inhibitors, administering the organic nitrate compounds and nitrosated and/or nitrosylated COX-2 selective inhibitors, or administering the organic nitrate compounds, COX-2 selective inhibitors and nitrosated and/or nitrosylated COX-2 selective inhibitors. The organic nitrate compounds and COX-2 selective inhibitors can be administered separately or as components of the same composition in one or more pharmaceutically acceptable carriers.

Yet another embodiment of the invention provides methods for improving the gastroprotective properties of H₂ receptor antagonists, increasing the rate of ulcer healing, decreasing the rate of recurrence of ulcers, treating inflammations, treating ophthalmic diseases and disorders, treating microbial infections, decreasing or reversing gastrointestinal toxicity and facilitating ulcer healing resulting from the administration of nonsteroidal antiinflammatory drugs (NSAIDs); improving the gastroprotective properties, anti-Helicobacter properties and antacid properties of H₂ receptor antagonists, preventing or treating gastrointestinal disorders, treating multiple sclerosis, treating ophthalmic diseases and disorders; and methods for treating viral infections, such as HIV disease, in a patient in need thereof which comprises administering to the patient a therapeutically effective amount of at least one organic nitrate compound comprising at least one sulfur atom and/or at least one disulfide group. The methods can optionally further comprise the administration of at least one H₂ receptor antagonist compound that is optionally substituted with at least one NO and/or NO₂ group (i.e., nitrosylated and/or nitrosated). In this embodiment of the invention, the methods can involve administering the organic nitrate compounds, administering the organic nitrate compounds and H₂ receptor antagonist compounds, administering the organic nitrate compounds and nitrosated H₂ receptor antagonist compounds, administering the organic nitrate compounds and nitrosylated H₂ receptor antagonist compounds, administering the organic nitrate compounds and nitrosated and/or nitrosylated H₂ receptor antagonist compounds, or administering the organic nitrate compounds, H₂ receptor antagonist compounds and nitrosated and/or nitrosylated H₂ receptor antagonist compounds. The organic nitrate compounds and H₂ receptor antagonist compounds can be administered separately or as components of the same composition in one or more pharmaceutically acceptable carriers.

Yet another embodiment of the invention provides methods for treating and/or gastrointestinal disorders; facilitating ulcer healing; decreasing the recurrence of ulcers; improving gastroprotective properties, anti-Helicobacter pylori properties or antacid properties of proton pump inhibitors; decreasing or reducing the gastrointestinal toxicity associated with the use of nonsteroidal antiinflammatory compounds and/or selective COX-2 inhibitors; treating and/or preventing bacterial infections and/or viral infections in a patient in need thereof which comprises administering to the patient a therapeutically effective amount of at least one organic nitrate compound comprising at least one sulfur atom and/or at least one disulfide group. The methods can optionally further comprise the administration of at least one proton pump inhibitor that is optionally substituted with at least one NO and/or NO₂ group (i.e., nitrosylated and/or nitrosated). In this embodiment of the invention, the methods can involve administering the organic nitrate compounds, administering the organic nitrate compounds and proton pump inhibitors, administering the organic nitrate compounds and nitrosated proton pump inhibitors, administering the organic nitrate compounds and nitrosylated proton pump inhibitors, administering the organic nitrate compounds and nitrosated and/or nitrosylated proton pump inhibitors, or administering the organic nitrate compounds, proton pump inhibitors and nitrosated and/or nitrosylated proton pump inhibitors. The organic nitrate compounds and proton pump inhibitors can be administered separately or as components of the same composition in one or more pharmaceutically acceptable carriers.

Yet another embodiment of the invention provides methods for treating and/or preventing sexual dysfunctions and/or enhancing sexual responses in a patient in need thereof which comprises administering to the patient a therapeutically effective amount of at least one organic nitrate compound comprising at least one sulfur atom and/or at least one disulfide group. The methods can optionally further comprise the administration of at least one vasoactive agent that is optionally substituted with at least one NO and/or NO₂ group (i.e., nitrosylated and/or nitrosated). In this embodiment of the invention, the methods can involve administering the organic nitrate compounds, administering the organic nitrate compounds and vasoactive agents, administering the organic nitrate compounds and nitrosated vasoactive agents, administering the organic nitrate compounds and nitrosylated vasoactive agents, administering the organic nitrate compounds and nitrosated and/or nitrosylated vasoactive agents, or administering the organic nitrate compounds, vasoactive agents and nitrosated and/or nitrosylated vasoactive agents. The organic nitrate compounds and vasoactive agents can be administered separately or as components of the same composition in one or more pharmaceutically acceptable carriers.

The invention also provides methods for treating and/or preventing diseases induced by the increased metabolism of cyclic guanosine 3′,5′-monophosphate (cGMP), such as hypertension, pulmonary hypertension, congestive heart failure, myocardial infraction, stable, unstable and variant (Prinzmetal) angina, atherosclerosis, cardiac edema, renal insufficiency, nephrotic edema, hepatic edema, stroke, asthma, bronchitis, chronic obstructive pulmonary disease (COPD), cystic fibrosis, dementia, immunodeficiency, premature labor, dysmenorrhoea, benign prostatic hyperplasis (BPH), bladder outlet obstruction, incontinence, conditions of reduced blood vessel patency, e.g., postpercutaneous transluminal coronary angioplasty (post-PTCA), peripheral vascular disease, allergic rhinitis, cystic fibrosis, and glucoma, and diseases characterized by disorders of gut motility, e.g., irritable bowel syndrome (IBS) in a patient in need thereof which comprises administering to the patient a therapeutically effective amount of at least one organic nitrate compound comprising at least one sulfur atom and/or at least one disulfide group. The methods can optionally further comprise the administration of at least one phosphodiesterase inhibitor that is optionally substituted with at least one NO and/or NO₂ group (i.e., nitrosylated and/or nitrosated). In this embodiment of the invention, the methods can involve administering the organic nitrate compounds, administering the organic nitrate compounds and phosphodiesterase inhibitors, administering the organic nitrate compounds and nitrosated phosphodiesterase inhibitors, administering the organic nitrate compounds and nitrosylated phosphodiesterase inhibitors, administering the organic nitrate compounds and nitrosated and/or nitrosylated phosphodiesterase inhibitors, or administering the organic nitrate compounds, phosphodiesterase inhibitors and nitrosated and/or nitrosylated phosphodiesterase inhibitors. The organic nitrate compounds and phosphodiesterase inhibitors can be administered separately or as components of the same composition in one or more pharmaceutically acceptable carriers.

Yet another embodiment of the invention provides methods for treating and/or preventing benign prostatic hyperplasia, variant (Printzmetal) angina, glaucoma, neurodegenerative disorders, vasospastic diseases, cognitive disorders, urge incontinence, or overactive bladder, or to reverse the state of anesthesia in a patient in need thereof which comprises administering to the patient a therapeutically effective amount of at least one organic nitrate compound comprising at least one sulfur atom and/or at least one disulfide group. The methods can optionally further comprise the administration of at least one α-adrenergic receptor antagonist that is optionally substituted with at least one NO and/or NO₂ group (i.e., nitrosylated and/or nitrosated). In this embodiment of the invention, the methods can involve administering the organic nitrate compounds, administering the organic nitrate compounds and α-adrenergic receptor antagonists, administering the organic nitrate compounds and nitrosated α-adrenergic receptor antagonists, administering the organic nitrate compounds and nitrosylated α-adrenergic receptor antagonists, administering the organic nitrate compounds and nitrosated and/or nitrosylated α-adrenergic receptor antagonists, or administering the organic nitrate compounds, α-adrenergic receptor antagonists and nitrosated and/or nitrosylated α-adrenergic receptor antagonists. The organic nitrate compounds and α-adrenergic receptor antagonists can be administered separately or as components of the same composition in one or more pharmaceutically acceptable carriers.

In another embodiment the invention provides methods for treating and/or preventing respiratory disorders, in a patient in need thereof which comprises administering to the patient a therapeutically effective amount of at least one organic nitrate compound comprising at least one sulfur atom and/or at least one disulfide group. The methods can optionally further comprise the administration of at least one steroid, β-agonist, anticholinergic, mast cell stabilizer or PDE inhibitor, that is optionally substituted with at least one NO and/or NO₂ group (i.e., nitrosylated and/or nitrosated). In this embodiment of the invention, the methods can involve administering the organic nitrate compounds, administering the organic nitrate compounds and steroids, β-agonists, anticholinergics, mast cell stabilizers or PDE inhibitors, administering the organic nitrate compounds and nitrosated steroids, nitrosated β-agonists, nitrosated anticholinergics, nitrosated mast cell stabilizers or nitrosated PDE inhibitors, administering the organic nitrate compounds and nitrosylated steroids, nitrosylated β-agonists, nitrosylated anticholinergics, nitrosylated mast cell stabilizers or nitrosylated PDE inhibitor, administering the organic nitrate compounds and nitrosated and/or nitrosylated steroids, nitrosated and/or nitrosylated β-agonists, nitrosated and/or nitrosylated anticholinergisc, nitrosated and/or nitrosylated mast cell stabilizers or nitrosated and/or nitrosylated PDE inhibitors, or administering the organic nitrate compounds, steroids, β-agonists, anticholinergics, mast cell stabilizers or PDE inhibitors and nitrosated and/or nitrosylated steroids, β-agonists, anticholinergics, mast cell stabilizers or PDE inhibitors. The organic nitrate compounds, steroids, β-agonists, anticholinergics, mast cell stabilizers and PDE inhibitors can be administered separately or as components of the same composition in one or more pharmaceutically acceptable carriers.

Yet another embodiment of the invention provides methods for treating and/or preventing restenosis in a patient in need thereof which comprises administering to the patient a therapeutically effective amount of at least one organic nitrate compound comprising at least one sulfur atom and/or at least one disulfide group. The methods can optionally further comprise the administration of at least one steroid, taxane, rapamycin, or tranilast, that is optionally substituted with at least one NO and/or NO₂ group (i.e., nitrosylated and/or nitrosated). In this embodiment of the invention, the methods can involve administering the organic nitrate compounds, administering the organic nitrate compounds and steroids, taxanes, rapamycins or tranilasts, administering the organic nitrate compounds and nitrosated steroids, nitrosated taxanes, nitrosated rapamycins or nitrosated tranilasts, administering the organic nitrate compounds and nitrosylated steroids, nitrosylated taxanes, nitrosylated rapamycins or nitrosylated tranilasts, administering the organic nitrate compounds and nitrosated and/or nitrosylated steroids, nitrosated and/or nitrosylated taxanes, nitrosated and/or nitrosylated rapamycins or nitrosated and/or nitrosylated tranilasts, administering the organic nitrate compounds and steroids, taxanes, rapamycins or tranilasts and nitrosated and/or nitrosylated steroids, taxanes, rapamycins or tranilasts. The organic nitrate compounds, steroids, taxanes, rapamycins or tranilasts can be administered separately or as components of the same composition in one or more pharmaceutically acceptable carriers.

Yet another embodiment of the invention provides compositions and methods for making compositions which contain at least one organic nitrate compound comprising at least one sulfur atom and/or at least one disulfide group, and, optionally, at least one steroid, taxane, rapamycin, steroid, tranilast, that is optionally substituted with at least one NO and/or NO₂ group (i.e., nitrosylated and/or nitrosated), that are bound to a natural or synthetic matrix, which can be applied with specificity to a biological site of interest. For example, the matrix containing the organic nitrate compound can be used to coat the surface of a medical device or instrument that comes into contact with blood (including blood components, blood products and the like) or vascular tissue.

Yet another embodiment of the invention provides methods for treating and/or preventing neurological disorders in a patient in need thereof which comprises administering to the patient a therapeutically effective amount of at least one organic nitrate compound comprising at least one sulfur atom and/or at least one disulfide group.

Yet another embodiment of the invention describes compositions and kits comprising at least one organic nitrate compound comprising at least one sulfur atom and/or at least one disulfide group, and, optionally, at least one NSAID, COX-2 inhibitor, H₂ receptor antagonist, proton pump inhibitor, vasoactive agent, steroid, β-agonist, anticholinergic, mast cell stabilizer, PDE inhibitor, taxane, rapamycin, tranilast, that is optionally substituted with at least one NO and/or NO₂ group (i.e., nitrosylated and/or nitrosated).

These and other aspects of the present invention are described in detail herein.

### DETAILED DESCRIPTION OF THE INVENTION

As used throughout the disclosure, the following terms, unless otherwise indicated, shall be understood to have the following meanings.

“NSAID” refers to a nonsteroidal anti-inflammatory compound or a nonsteroidal anti-inflammatory drug. NSAIDs inhibit cyclooxygenase, the enzyme responsible for the biosyntheses of the prostaglandins and certain autocoid inhibitors, including inhibitors of the various isoezymes of cyclooxygenase (including but not limited to cyclooxygenase-1 and -2), and as inhibitors of both cyclooxygenase and lipoxygenase.

“Cyclooxygenase-2 (COX-2) inhibitor” refers to a compound that selectively inhibits the cyclooxygenase-2 enzyme over the cyclooxygenase-1 enzyme. Preferably, the compound has a cyclooxygenase-2 IC₅₀ of less than about 0.5 μM, and also has a selectivity ratio of cyclooxygenase-2 inhibition over cyclooxygenase-1 inhibition of at least 50, and more preferably of at least 100. Even more preferably, the compound has a cyclooxygenase-1 IC₅₀ of greater than about 1 μM, and more preferably of greater than 20 μM. The compound can also inhibit the enzyme, lipoxygenase and/or phosphodiestase. Such preferred selectivity may indicate an ability to reduce the incidence of common NSAID-induced side effects.

“Nitric oxide adduct” or “NO adduct” refers to compounds and functional groups which, under physiological conditions, can donate, release and/or directly or indirectly transfer any of the three redox forms of nitrogen monoxide (NO⁺, NO⁻, NO•), such that the biological activity of the nitrogen monoxide species is expressed at the intended site of action.

“Nitric oxide releasing” or “nitric oxide donating” refers to methods of donating, releasing and/or directly or indirectly transferring any of the three redox forms of nitrogen monoxide (NO⁺, NO—, NO•), such that the biological activity of the nitrogen monoxide species is expressed at the intended site of action.

“Nitric oxide donor” or “NO donor” refers to compounds that donate, release and/or directly or indirectly transfer a nitrogen monoxide species, and/or stimulate the endogenous production of nitric oxide or endothelium-derived relaxing factor (EDRF) in vivo and/or elevate endogenous levels of nitric oxide or EDRF in vivo. “NO donor” also includes compounds that are substrates for nitric oxide synthase.

“Gastrointestinal disorder” refers to any disease or disorder of the upper gastrointestinal tract of a patient including, for example, inflammatory bowel disease, Crohn's disease, gastritis, irritable bowel syndrome, constipation, ulcerative colitis, peptic ulcers, stress ulcers, gastric hyperacidity, dyspepsia, gastroparesis, Zollinger-Ellison syndrome, gastroesophageal reflux disease, bacterial infections (including, for example, a Helicobacter Pylori associated disease), short-bowel (anastomosis) syndrome, hypersecretory states associated with systemic mastocytosis or basophilic leukemia and hyperhistaminemia, and bleeding peptic ulcers that result, for example, from neurosurgery, head injury, severe body trauma or burns.

“Upper gastrointestinal tract” refers to the esophagus, the stomach, the duodenum and the jejunum.

“Ulcers” refers to lesions of the upper gastrointestinal tract lining that are characterized by loss of tissue. Such ulcers include gastric ulcers, duodenal ulcers and gastritis.

“Inflammatory disease states and disorders” refers to reperfusion injury to an ischemic organ (e.g., reperfusion injury to the ischemic myocardium), myocardial infarction, inflammatory bowel disease, rheumatoid arthritis, osteoarthritis, psoriasis, organ transplant rejection, inflammation of the ear, eye, throat, nose or skin, organ preservation, a female or male sexual dysfunction, radiation-induced injury, asthma, respiratory disorder, metastasis, influenza, incontinence, stroke, burn, trauma, acute pancreatitis, pyelonephritis, hepatitis, an autoimmune disease, an immunological disorder, senile dementia, insulin-dependent diabetes mellitus, disseminated intravascular coagulation, fatty embolism, Alzheimer's disease, adult or infantile respiratory disease, carcinogenesis or a hemorrhage in a neonate, restenosis, atherosclerosis, atherogenesis, angina, (particularly chronic, stable angina pectoris), ischemic disease, congestive heart failure or pulmonary edema associated with acute myocardial infarction, thrombosis, thromboemboembolic events, hypertension (especially hypertension associated with cardiovascular surgical procedures), platelet aggregation, platelet adhesion, smooth muscle cell proliferation, vascular complications associated with the use of medical devices, wounds associated with the use of medical devices, cerebrovascular ischemic events, and the like. Complications associated with the use of medical devices may occur as a result of increased platelet deposition, activation, thrombus formation or consumption of platelets and coagulation proteins. Such complications, include, for example, myocardial infarction, ischemic stroke, transient ischemic stroke, thromboemboembolic events, pulmonary thromboembolism, cerebral thromboembolism, thrombophlebitis, thrombocytopenia, bleeding disorders and/or any other complications which occur either directly or indirectly as a result of the foregoing disorders.

“Restenosis” is a cardiovascular disease or disorder that refers to the closure of a peripheral or coronary artery following trauma to the artery caused by an injury such as, for example, angioplasty, balloon dilation, atherectomy, laser ablation treatment or stent insertion. For these angioplasty procedures, restenosis occurs at a rate of about 30-60% depending upon the vessel location, lesion length and a number of other variables. Restenosis can also occur following a number of invasive surgical techniques, such as, for example, transplant surgery, vein grafting, coronary artery bypass surgery, endarterectomy, heart transplantation, balloon angioplasty, atherectomy, laser ablation, endovascular stenting, and the like.

“Atherosclerosis” is a form of chronic vascular injury in which some of the normal vascular smooth muscle cells in the artery wall, which ordinarily control vascular tone regulating blood flow, change their nature and develop “cancer-like” behavior. These vascular smooth muscle cells become abnormally proliferative, secreting substances such as growth factors, tissue-degradation enzymes and other proteins, which enable them to invade and spread into the inner vessel lining, blocking blood flow and making that vessel abnormally susceptible to being completely blocked by local blood clotting, resulting in the death of the tissue served by that artery. Atherosclerotic cardiovascular disease, coronary heart disease (also known as coronary artery disease or ischemic heart disease), cerebrovascular disease and peripheral vessel disease are all common manifestations of atherosclerosis and are therefore encompassed by the terms “atherosclerosis” and “atherosclerotic disease”.

“Thromboemboembolic events” includes, but is not limited to, ischemic stroke, transient ischemic stroke, myocardial infarction, angina pectoris, thrombosis, thromboembolism, thrombotic occlusion and reocclusion, acute vascular events, restenosis, transient ischemic attacks, and first and subsequent thrombotic stroke. Patients who are at risk of developing thromboembolic events, may include those with a familial history of, or genetically predisposed to, thromboembolic disorders, who have had ischemic stroke, transient ischemic stroke, myocardial infarction, and those with unstable angina pectoris or chronic stable angina pectoris and patients with altered prostacyclin/thromboxane A₂ homeostasis or higher than normal thromboxane A₂ levels leading to increase risk for thromboembolism, including patients with diabetes and rheumatoid arthritis.

“Ophthalmic diseases and disorders” refers to any disease or disorder of the eye. Ophthalmic diseases and disorders include, but are not limited to, glaucoma, inflammation of the eye and elevation of intraocular pressure, and the like

“H₂ receptor antagonist” refers to any compound that reversibly or irreversibly blocks the activation of any H₂ receptor.

“Proton pump inhibitor” refers to any compound that reversibly or irreversibly blocks gastric acid secretion by inhibiting the H⁺/K⁺-ATPase enzyme system at the secretory surface of the gastric parietal cell.

“Viral infection” refers to both RNA and DNA viral infections. The RNA viral infections include, but are not limited to, orthomyxoviridae, paramyxoviridae, picornaviridae, rhabdoviridae, coronavaridae, togaviridae, bunyaviridae, arenaviridae and reteroviridae. The DNA viral infections include, but are not limited to, adenoviridae, proxyiridae, papovaviridae, herpetoviridae and herpesviridae. The most preferable viral infections are those of the herpetoviridae family, such as, for example, herpes simplex viruses HSV-1 and HSV-2, cytomegalovirus (CMV), herpes varicella-zoster (VZV), Epstein-Barr (EBV), HHV6, HHV7, pseudorabies and rhinotracheitis, and the like.

“Vasoactive agent” refers to any therapeutic agent capable of relaxing vascular and/or nonvascular smooth muscle. Suitable vasoactive agents include, but are not limited to, potassium channel activators, calcium channel blockers, β-blockers, long and short acting α-adrenergic receptor antagonists, prostaglandins, phosphodiesterase inhibitors, adenosine, ergot alkaloids, vasoactive intestinal peptides, dopamine agonists, opioid antagonists, endothelin antagonists, thromboxane inhibitors and the like.

“Phosphodiesterase inhibitor” or “PDE inhibitor” refers to any compound that inhibits the enzyme phosphodiesterase. The term refers to selective or non-selective inhibitors of cyclic guanosine 3′,5′-monophosphate phosphodiesterases (cGMP-PDE) and cyclic adenosine 3′,5′-monophosphate phosphodiesterases (cAMP-PDE).

“α-adrenergic receptor antagonists” refers to any compound that reversibly or irreversibly blocks the activation of any α-adrenergic receptor.

“Thromboxane inhibitor” refers to any compound that reversibly or irreversibly inhibits thromboxane synthesis, and includes compounds which are the so-called thromboxane A₂ receptor antagonists, thromboxane A₂ antagonists, thromboxane A₂/prostaglandin endoperoxide antagonists, thromboxane receptor (TP) antagonists, thromboxane antagonists, thromboxane synthase inhibitors, and dual acting thromboxane synthase inhibitors and thromboxane receptor antagonists.

“Thromboxane A₂ receptor antagonist” refers to any compound that reversibly or irreversibly blocks the activation of any thromboxane A₂ receptor.

“Thromboxane synthase inhibitor” refers to any compound that reversibly or irreversibly inhibits the enzyme thromboxane synthesis thereby reducing the formation of thromboxane A₂.

“Dual acting thromboxane receptor antagonist and thromboxane synthase inhibitor” refers to any compound that simultaneously acts as a thromboxane A₂ receptor antagonist and a thromboxane synthase inhibitor.

“Taxane” refers to any compound that contains the carbon core framework represented by formula A:

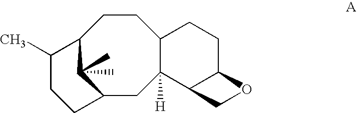

“Sexual dysfunction” refers to any sexual dysfunction in a patient, including, for example, sexual desire disorders, sexual arousal disorders, orgasmic disorders and sexual pain disorders.

“Female sexual dysfunction” refers to any female sexual dysfunction including, for example, sexual desire disorders, sexual arousal dysfunctions, orgasmic dysfunctions, sexual pain disorders, dyspareunia, and vaginismus. The female can be pre-menopausal or menopausal.

“Male sexual dysfunction” refers to any male sexual dysfunction including, for example, male erectile dysfunction and impotence.

“Pathological conditions resulting from abnormal cell proliferation” refers to any abnormal cellular proliferation of malignant or non-malignant cells in various tissues and/or organs, including but not limited to, muscle, bone, conjunctive tissues, skin, brain, lungs, sexual organs, lymphatic system, renal system, mammary cells, blood cells, liver, the digestive system, pancreas, thyroid, adrenal glands and the like. These pathological conditions can also include psoriasis; solid tumors; ovarian, breast, brain, prostate, colon, osesophageal, lung, stomach, kidney and/or testicular cancer; Karposi's sarcoma, cholangiocarcinoma; choriocarcinoma; neoblastoma; Wilm's tumor; Hodgkin's disease; melanomas; multiple myelomas; chronic lymphocytic leukemias, and acute or chronic granulocytic lymphomas.

“Artificial surface” refers to any synthetic material contained in a device or apparatus that is in contact with blood, vasculature or other tissues.

“Blood” includes blood products, blood components and the like.

“Platelet adhesion” refers to the contact of a platelet with a foreign surface, including any artificial surface, such as a medical device or instrument, as well as an injured vascular surfaces, such as collagen. Platelet adhesion does not require platelet activation. Unactivated, circulating platelets will adhere to injured vascular surfaces or artificial surfaces via binding interactions between circulating von Willdebrand factor and platelet surface glycoprotein Ib/IX.

“Platelet aggregation” refers to the binding of one or more platelets to each other. Platelet aggregation is commonly referred to in the context of generalized atherosclerosis, not with respect to platelet adhesion on vasculature damaged as a result of physical injury during a medical procedure. Platelet aggregation requires platelet activation which depends on the interaction between the ligand and its specific platelet surface receptor.

“Passivation” refers to the coating of a surface which renders the surface non-reactive.

“Platelet activation” refers either to the change in conformation (shape) of a cell, expression of cell surface proteins (e.g., the IIb/IIIa receptor complex, loss of GPIb surface protein), and secretion of platelet derived factors (e.g., serotonin, growth factors).

“Respiratory disorders” refers to disorders such as, asthma, chronic obstructive pulmonary disease (COPD), cystic fibrosis, pneumonia, traumatic injury, aspiration or inhalation injury, fat embolism in the lung, acidosis inflammation of the lung, adult respiratory distress syndrome, acute pulmonary edema, acute mountain sickness, post-cardiac surgery, acute pulmonary hypertension, persistent pulmonary hypertension of the newborn, perinatal aspiration syndrome, hyaline membrane disease, acute pulmonary thromboembolism, herapin-protamine reactions, sepsis, status asthmaticus or hypoxia (including iatrogenic hypoxia) and other forms of reversible pulmonary vasoconstriction.

“Neurological disorder” refers to disorders such as, Parkinson's disease, Alzheimer's disease, Huntington disease, multiple sclerosis, amylotrophic lateral sclerosis, AID-induced dementia, epilepsy, trauma to the head, cognitive disorders, memory loss, dementia, and the like.

“Patient” refers to animals, preferably mammals, more preferably humans, and includes children and adults.

“Therapeutically effective amount” refers to the amount of the compound and/or composition that is effective to achieve its intended purpose.

“Medical device” refers to any intravascular or extravascular medical devices, medical instruments, foreign bodies and the like. Examples of intravascular medical devices and instruments include balloons or catheter tips adapted for insertion, prosthetic heart valves, sutures, synthetic vessel grafts, stents (e.g. Palmaz-Schatz stent), drug pumps, arteriovenous shunts, artificial heart valves, artificial implants, foreign bodies introduced surgically into the blood vessels or at vascular sites, leads, pacemakers, implantable pulse generators, implantable cardiac defibrillators, cardioverter defibrillators, defibrillators, spinal stimulators, brain stimulators, sacral nerve stimulators, chemical sensors, and the like. Examples of extravascular medical devices and instruments include plastic tubing, dialysis bags or membranes whose surfaces come in contact with the blood stream of a patient.

“Transdermal” refers to the delivery of a compound by passage through the skin and into the blood stream.

“Transmucosal” refers to delivery of a compound by passage of the compound through the mucosal tissue and into the blood stream.

“Penetration enhancement” or “permeation enhancement” refers to an increase in the permeability of the skin or mucosal tissue to a selected pharmacologically active compound such that the rate at which the compound permeates through the skin or mucosal tissue is increased.

“Carriers” or “vehicles” refers to carrier materials suitable for compound administration and include any such material known in the art such as, for example, any liquid, gel, solvent, liquid diluent, solubilizer, or the like, which is non-toxic and which does not interact with any components of the composition in a deleterious manner.

The organic nitrate compounds of the invention comprising at least one sulfur atom and/or at least one disulfide group include, but are not limited to, those disclosed in U.S. Pat. Nos. 5,284,872, 5,428,061 and 5,661,129, and in Org Lett., 3: 1113-11i6 (2001); and in J. Pharmacol. Exp. Ther., 287:527-537 (1998) and in J. Pharmacol. Exp. Ther., 206: 818-823 (2000), the disclosure of each of which are incorporated by reference herein in their entirety.

In one embodiment, the invention describes the methods of use of organic nitrate compound comprises at least one disulfide group of Formula (I), and pharmaceutically acceptable salts thereof:

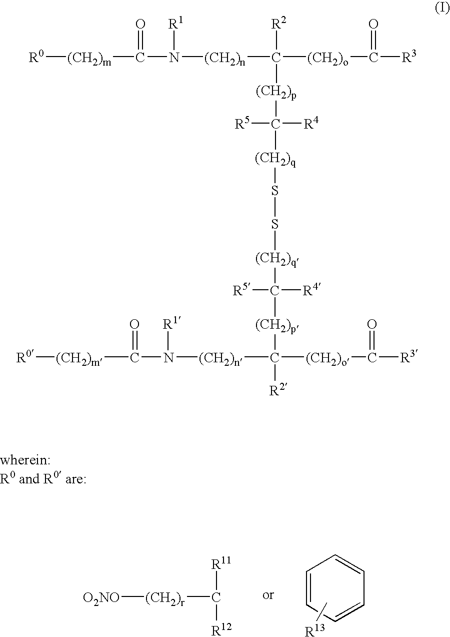

R¹¹ is hydrogen, an alkyl group having 1 to 6 carbon atoms, a substituted lower alkyl wherein the substituent is halogen, hydroxyl, lower alkoxy, aryloxy, amino, lower alkylamino, acylamino, acyloxy, arylamino, mercapto, lower alkylthio or arylthio,

R¹² is R¹¹ hydrogen or a lower alkyl group;

R¹³ is a nitratoalkyl group having 1 to 6 carbon atoms;

r is an integer from 0 to 10;

R¹ and R^{1′} are each independently hydrogen or lower alkyl;

R² and R^{2′} are each independently hydrogen, lower alkyl, phenyl, methoxyphenyl, phenyl-lower-alkyl, methoxyphenyl-lower-alkyl, hydroxyphenyl-lower-alkyl, hydroxy-lower-alkyl, alkoxy-lower-alkyl, amino-lower-alkyl, acylamino-lower-alkyl, mercapto-lower-alkyl or lower alkylthio-lower-alkyl;

R³ and R^{3′} are each independently hydroxyl, lower alkoxy, lower alkenoxy, di-lower-alkylamino-lower-alkoxy, acylamino-lower-alkoxy, acyloxy-lower-alkoxy, aryloxy, aryl-lower-alkoxy, substituted aryloxy or substituted aryl-lower-alkoxy, in which the substituent is methyl, halogen or methoxy; amino, lower alkylamino, di-lower-alkylamino, aryl-lower-alkylamino, hydroxy-lower-alkyl-amino, pyrrolidine, piperidine, morpholine, piperazine or amino-acid residues via peptide linkage;

R⁴ and R^{4′} are each independently hydrogen or lower alkyl;

R⁵ and R^{5′} are each independently R⁴, R^{4′} hydrogen or lower alkyl;

R² and R³, and R^{2′} and R^{3′}, can be linked together to form an ester or an amide;

R¹ and R², and R^{1′} and R^{2′}, can be linked together to form an alkylene bridge having 2 to 4 carbon atoms, an alkylene bridge having 2 to 3 carbon atoms and a sulfur atom, an alkylene bridge having 3 to 4 carbon atoms, which contains a double bond or an alkylene bridge, optionally substituted by hydroxyl, lower alkoxy, lower alkyl or di-lower-alkyl;

m, n, o, p, q, m′, n′, o′, p′ and q′ are each independently integers from 0 to 10;

Another embodiment of the invention describes organic nitrate compound comprises at least one sulfur atom of Formula (II) and pharmaceutically acceptable salts thereof:

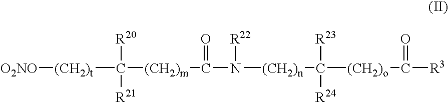

wherein:

R²⁰ and R²¹ are each independently a hydrogen, an alkyl having 1 to 6 carbon atoms, a substituted lower alkyl in which the substituent is a halogen, groups defined by R³ containing hydroxy, lower alkoxy, aryloxy, amino, lower alkylamino, acylamino, acyloxy, arylamino, mercapto, lower alkylthio or arylthio;

R²² is hydrogen or lower alkyl;

R²³ is hydrogen, lower alkyl, phenyl, methoxy phenyl, phenyl-lower alkyl, methoxyphenyl-lower alkyl, hydroxyphenyl-lower alkyl, hydroxy-lower alkyl, alkoxy-lower alkyl, amino-lower alkyl, acylamino-lower alkyl, mercapto-lower alkyl or lower alkylthio-lower alkyl;

R²⁴ is lower alkylthiol, —SH, S-acyl compound of lower alkylthiol, preferably —S-acetyl, —S-propionyl, —S-butyryl, —S-isobutyryl, —S-capryl, —S-pivaloyl, —S-benzoyl;

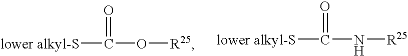

and lower alkylthio-lower alkanoic acid and esters and amides thereof, and lower alkylthio-lower alkyl;

R²⁵ is hydrogen and lower alkyl groups in which R³ and R²⁴ are bonded together and form part of a thiolactone group, groups in which R³ and R²³ are bonded together in the form of an ester or amide, groups in which R²² and R²³ are bonded together in the form of an alkylene bridge with 2 to 4 carbon atoms, an alkylene bridge with 2 to 3 carbon atoms and a sulfur atom, an alkylene bridge with 3 to 4 carbon atoms, which contains a double bond or an alkylene bridge as above, which can be substituted by one or more hydroxy, lower alkoxy, lower alkyl or di-lower alkyl groups; and

R³, m, n, and o are as defined herein.

In other embodiments of the invention, the compounds of Formula (I) are asymmetric disulphides containing sulfur-containing amino acids, preferably glutathione or penicillamine; the aliphatic part(s) of the nitratoalkylarylalkanoic acid and nitratoalkanoic acid constituents has (have) a chain length of 2-6 carbon atoms; and optionally are straight-chain, branched, racemic or optically isomeric; the nitratoalkane- and nitratoalkylarylalkanecarboxylic acid derivatives of the compounds of Formula (I) contain disulphides of sulfur-containing amino acids, preferably cystine, homocystine or penicillamine disulphide. The amino-acid disulphides are in the stereochemical L or DL form.

In yet other embodiments of the invention, the compounds of Formula (I) are:

N′-3-nitratopivaloyl-L-cysteinamide-glutathione mixed disulphide,

N′-3-nitratopivaloyl-L-cysteine ethyl ester-glutathione mixed disulphide;

N′-3-nitratopivaloyl-L-cysteine ethyl ester-N′-acetyl-L-cysteine mixed disulphide;

N-(3-nitratopivaloyl)-L-cysteine ethyl ester-D,L-penicillamine mixed disulphide;

2-acetylamino-3-(2-(2,2-dimethyl-3-nitrooxy-propionylamino)-2-ethoxycarbonyl ethyldisulphanyl)-3-methylbutyric acid;

N,N′-di(3-nitratopivaloyl)-L-cystine;

N,N′-di(3-nitratopivaloyl)-D,L-homocystine;

N,N′-di(3-nitratopivaloyl)-L-cystine diethyl ester;

N,N′-di(3-nitratopivaloyl)-D,L-homocystine diethyl ester;

N,N′-di(3-nitratopivaloyl)-L-cystine di-tert.-butyl ester;

N,N′-di(4-nitratomethylbenzoyl)-L-cystine dimethyl ester;

N,N′-di(3-nitratomethylbenzoyl)-L-cystine dimethyl ester;

N,N′-di(4-nitratomethylbenzoyl)-L-cystine-di(N,N′-butylamide);

N,N′-di(3-nitratomethylbenzoyl)-L-cystine-di(N,N′-butylamide);

N,N′-di(4-nitratomethylbenzoyl)-L-cystinediamide;

N,N′-di(3-nitratomethylbenzoyl)-L-cystinediamide;

N,N′-di(3-nitratopivaloyl)-L-penicillamine disulphidediamide;

N,N′-di(3-nitratopivaloyl)-L-cystinediamide;

N,N′-di(3-nitratopivaloyl)-L-cystine-di(N,N′-methylamide);

N,N′-di(3-nitratopivaloyl)-L-cystine-di(N,N′-butylamide);

N,N′-di(3-nitratopivaloyl)-L-cystine-di(N,N′-tertiary-butylamide);

N,N′-di(3-nitratopivaloyl)-L-cystine-dimorpholide;

N,N′-di(3-nitratopivaloyl)-L-cystinediisopropyl ester, and pharmaceutically acceptable salts thereof.

In other embodiments of the invention, in the compounds of Formula (II) the nitrato alkanoic acid components can have a chain length of 2 to 6, and optionally maybe straight-chain or branched chain, racemic or optically isomeric; the amino acid is glycine, N-acetylglycine, alanine, N-acetylalanine, arginine, N-acetylarginine, N-α-benzoylarginine, cysteine, N-acetylcysteine, N,S-dipivaloylcysteine, cystine, N,N-diacetylcystine, leucine, N-acetylleucine, lysine, N-α-acetyllysine, N-ε-acetyllysine, N-α-ε-diacetyllysine, proline, N-acetylproline, serine, N-acetylserine, O-acetylserine, N,O-diacetylserine, methionine, N-benzoylmethionine, phenylalanine, N-benzoylphenylalanine, N-acetylphenylalanine, asparagine, N-acetylasparagine, N-acetylasparagine monoethyl ester, glutamic acid or N-acetylglutamic acid monomethyl ester, and more preferably, the amino acid is cysteine, methionine or homocysteine; and even more preferably the cysteine is its methyl, ethyl or propyl ester; the —SH group of cysteine is preferably esterified with a lower alkanoic acid having 2 to 8 carbon atoms. The amino-acids are in the stereochemical L or DL form.

In yet other embodiments the compounds of Formula (II) are:

N-nitrato-pivaloyl-S-(N-acetyl-glycyl)-L-cysteine ethyl ester (compound SPM 5186);

N-nitrato-pivaloyl-S-(N-acetyl-alanyl)-L-cysteine ethyl ester (compound SPM 5185);

N-nitrato-pivaloyl-S-(N-acetyl-leucyl)-L-cysteine ethyl ester. N-(2-nitratoacetyl)-cysteine ethyl ester;

N-(2-nitratoacetyl)-S-acetyl-cysteine ethyl ester;

N-(2-nitratoacetyl)-S-propionyl-cysteine ethyl ester;

N-(2-nitratoacetyl)-S-pivaloyl-cysteine ethyl ester;

N-(2-nitratoacetyl)-methionine methyl ester;

N-(2-nitratopropionyl)-cysteine;

N-(2-nitratopropionyl)-cysteine ethyl ester;

N-(2-nitratopropionyl)-methionine ethyl ester;

N-(2-nitratobutyryl)-cysteine;

N-(2-nitratobutyryl)-cysteine ethyl ester;

N-(2-nitratobutyryl)-S-acetyl-cysteine ethyl ester;

N-(2-nitratobutyryl)-S-butyryl-cysteine ethyl ester;

N-(2-nitratobutyryl)-methionine ethyl ester;

N-(2-nitratoisobutyryl)-cysteine;

N-(2-nitratoisobutyryl)-cysteine ethyl ester;

N-(2-nitratoisobutyryl)-S-benzoyl-cysteine ethyl ester;

N-(2-nitratoisobutyryl)-S-acetyl-cysteine ethyl ester;

N-(2-nitratoisobutyryl)-S-pivaloyl-cysteine ethyl ester;

N-(2-nitratoisobutyryl)-methionine ethyl ester;

N-(3-nitratobutyryl)-cysteine;

N-(3-nitratobutyryl)-cysteine ethyl ester;

N-(3-nitratobutyryl)-S-acetyl-cysteine ethyl ester;

N-(3-nitratobutyryl)-S-propionyl-cysteine ethyl ester;

N-(3-nitratobutyryl)-methionine ethyl ester;

N-(3-nitratobutyryl)-homocysteine thiolactone;

N-(3-nitratopivaloyl)-cysteine;

N-(3-nitratopivaloyl)-cysteine ethyl ester;

N-(3-nitratopivaloyl)-cysteine ethyl ester-S-ethyl carbonate;

N-(3-nitratopivaloyl)-S-acetyl-cysteine ethyl ester;

N-(3-nitratopivaloyl)-S-propionyl-cysteine ethyl ester;

N-(3-nitratopivaloyl)-S-butyryl-cysteine ethyl ester;

N-(3-nitratopivaloyl)-S-isobutyryl-cysteine ethyl ester;

N-(3-nitratopivaloyl)-S-pivaloyl-cysteine ethyl ester;

N-(3-nitratopivaloyl)-S-benzoyl-cysteine ethyl ester;

N-(3-nitratopivaloyl)-methionine ethyl ester;

N-(3-nitratopivaloyl)-methionine;

N-(3-nitratopivaloyl)-homocysteine thiolactone;

N-(2-nitratohexanoyl)-cysteine ethyl ester;

N-(2-nitratohexanoyl)-S-propionyl-cysteine ethyl ester;

N-(3-nitratohexanoyl)-cysteine ethyl ester;

N-(3-nitratohexanoyl)-methionine methyl ester;

N-(12-nitratolauroyl)-cysteine;

N-(12-nitratolauroyl)-cysteine ethyl ester;

N-(12-nitratolauroyl)-S-acetyl-cysteine;

N-(12-nitratolauroyl)-S-pivaloyl-cysteine;

compound SPM 3672; compound SPM 6373;

and esters thereof, preferably lower alkyl esters such as the methyl, propyl, isopropyl, butyl and pentyl esters; and pharmaceutically acceptable slats thereof.

Preferred organic nitrate compounds of the invention comprising at least one sulfur atom and/or at least one disulfide group include SP/W 5185, SP/W 5186, SP/M 6373 and SP/W 3672.

The compounds of Formula (I) and (II) can be prepared according to the procedures disclosed in U.S. Pat. Nos. 5,284,872, 5,428,061, 5,661,129, 5,807,847, and 5,883,122, and in Org Lett., 3: 1113-11i6 (2001); and in J. Pharmacol. Exp. Ther., 287:527-537 (1998) and in J. Pharmacol. Exp. Ther., 206: 818-823 (2000), the disclosures of each of which are incorporated by reference herein in their entirety.

The organic nitrate compounds of the invention comprising at least one sulfur atom and/or at least one disulfide group donate, transfer or release a biologically active form of nitrogen monoxide (i.e., nitric oxide). Nitrogen monoxide can exist in three forms: NO— (nitroxyl), NO• (uncharged nitric oxide) and NO⁺ (nitrosonium). NO• is a highly reactive short-lived species that is potentially toxic to cells. This is critical because the pharmacological efficacy of NO depends upon the form in which it is delivered. In contrast to the nitric oxide radical (NO•), nitrosonium (NO⁺) does not react with O₂ or O₂⁻ species, and functionalities capable of transferring and/or releasing NO⁺ and NO— are also resistant to decomposition in the presence of many redox metals. Consequently, administration of charged NO equivalents (positive and/or negative) is a more effective means of delivering a biologically active NO to the desired site of action.

The organic nitrate compounds of the present invention comprising at least one sulfur atom and/or at least one disulfide group, can be administered for treating, preventing and/or reducing inflammation, pain, and fever; for decreasing or reversing the gastrointestinal, renal and other toxicities resulting from the use of nonsteroidal antiinflammatory compounds; for treating and/or preventing gastrointestinal disorders; for facilitating wound healing; for treating inflammatory disease states and/or disorders; and for treating and/or preventing ophthalmic diseases and/or disorders; for treating and/or improving gastrointestinal properties of COX-2 selective inhibitors; for treating and/or preventing renal toxicity; for treating and/or preventing COX-2 mediated disorders; for decreasing the recurrence of ulcers; for improving gastroprotective properties, anti-Helicobacter pylori properties or antacid properties of proton pump inhibitors; treating and/or preventing bacterial infections, microbial infections, multiple sclerosis, and/or viral infections; for improving gastroprotective properties of H₂ receptor antagonists; for treating and/or preventing restenosis, autoimmune diseases, pathological conditions resulting from abnormal cell proliferation, polycystic kidney disease, inflammatory diseases or to inhibit wound contraction; for treating or preventing sexual dysfunctions in males and females, for enhancing sexual responses in males and females; for treating or preventing benign prostatic hyperplasia, hypertension, neurodegenerative disorders, vasospastic diseases, cognitive disorders, urge incontinence, and overactive bladder; for reversing the state of anesthesia; for treating or preventing diseases induced by the increased metabolism of cyclic guanosine 3′,5′-monophosphate (cGMP) and for treating respiratory disorders. The organic nitrate compounds of the present invention comprising at least one sulfur atom and/or at least one disulfide group can be optionally administered to a patient with at least one NSAID, COX-2 inhibitor, H₂ receptor antagonist, proton pump inhibitor, vasoactive agent, steroid, β-agonist, anticholinergic, mast cell stabilizer, PDE inhibitor, taxane, rapamycin, tranilast, that is optionally substituted with at least one NO and/or NO₂ group (i.e., nitrosylated and/or nitrosated) through one or more sites such as oxygen (hydroxyl condensation), sulfur (sulfhydryl condensation) and/or nitrogen. The nitrosated and/or nitrosylated NSAID, COX-2 inhibitor, H₂ receptor antagonist, proton pump inhibitor, vasoactive agent, steroid, β-agonist, anticholinergic, mast cell stabilizer, PDE inhibitor, taxane, rapamycin, tranilast, of the invention donate, transfer or release a biologically active form of nitrogen monoxide (i.e., nitric oxide). In one embodiment of the invention the diseases and disorders are preferably, inflammation, pain, gastrointestinal, restenosis, sexual dysfunctions, neurological conditions and respiratory diseases and disorders.

The methods for treating and/or preventing inflammation, pain and fever; decreasing and/or reversing gastrointestinal, renal and other toxicities resulting from the use of nonsteroidal antiinflammatory compounds; and treating and/or preventing gastrointestinal disorders, for treating inflammatory disease states and disorders, for treating and/or preventing ophthalmic diseases or disorders; in a patient in need thereof, include those disclosed in U.S. Pat. Nos. 5,703,073, 6,034,232, 6,043,232, 6,048,858, 6,051,588, 6,057,347, 6,083,515, 6,143,734, 6,297,260 and 6,323,234; and in WO 94/03421, WO 94/04484, WO 94/12463, WO 95/09831, WO 95/30641, WO 97/27749, WO 98/19672, WO 01/00563, WO 00/51988, WO 00/72838, WO 01/04082, WO 01/10814, WO 01/45703, WO 02/11707, and WO 02/30866, the disclosure of each of which are incorporated by reference herein in their entirety. In these methods the organic nitrate compounds comprising at least one sulfur atom and/or at least one disulfide group can optionally be administered with at least one NSAID that is optionally substituted with at least one NO and/or NO₂ group (i.e., nitrosylated and/or nitrosated).

Suitable NSAIDs, include, but are not limited to, acetaminophen, aspirin, diclofenac, ibuprofen, ketoprofen, naproxen and the like. Suitable NSAIDs are described more fully in the literature, such as in Goodman and Gilman, The Pharmacological Basis of Therapeutics (9th Edition), McGraw-Hill, 1995, Pgs. 617-657; and the Merck Index on CD-ROM, Twelfth Edition, Version 12:1, 1996. NSAIDs and their nitrosating and/or nitrosylated derivatives are also disclosed in U.S. Pat. Nos. 5,703,073, 6,034,232, 6,043,232, 6,048,858, 6,051,588, 6,057,347, 6,083,515, 6,143,734, 6,297,260 and 6,323,234; and in WO 94/03421, WO 94/04484, WO 94/12463, WO 95/09831, WO 95/30641, WO 97/27749, WO 98/19672, WO 01/00563, WO 00/51988, WO 00/72838, WO 01/04082, WO 01/10814, WO 01/45703, WO 02/11707 and WO 02/30866; the disclosure of each of which are incorporated by reference herein in their entirety.

The method for treating and/or improving the gastrointestinal properties of COX-2 inhibitors; for facilitating wound healing; for treating and/or preventing toxicity; and for treating and/or preventing COX-2 mediated disorders (i.e., disorders resulting from elevated levels of COX-2) include those disclosed in WO 01/46703, the disclosure of each of which are incorporated by reference herein in their entirety. In these methods the organic nitrate compounds comprising at least one sulfur atom and/or at least one disulfide group can optionally be administered with at least one COX-2 inhibitor that is optionally substituted with at least one NO and/or NO₂ group (i.e., nitrosylated and/or nitrosated).

Suitable COX-2 inhibitors include, but are not limited to, those disclosed in, for example, U.S. Pat. Nos. 5,134,142, .5,344,991, 5,380,738, 5,393,790, 5,409,944, 5,434,178, 5,436,265, 5,466,823, 5,474,995, 5,475,021, 5,486,534, 5,504,215, 5,508,426, 5,510,368, 5,510,496, 5,516,907, 5,521,207, 5,521,213, 5,536,752, 5,550,142, 5,552,422, 5,563,165, 5,580,985, 5,585,504, 5,596,008, 5,604,253, 5,604,260, 5,616,601, 5,620,999, 5,633,272, 5,639,780, 5,643,933, 5,677,318, 5,681,842, 5,686,460, 5,686,470, 5,691,374, 5,696,143, 5,698,584, 5,700,816, 5,710,140, 5,719,163, 5,733,909, 5,750,558, 5,753,688, 5,756,530, 5,756,531, 5,760,068, 5,776,967, 5,776,984, 5,783,597, 5,789,413, 5,807,873, 5,817,700, 5,824,699, 5,830,911, 5,840,746, 5,840,924, 5,849,943, 5,859,257, 5,861,419, 5,883,267, 5,905,089, 5,908,852, 5,908,858, 5,935,990, 5,945,539, 5,972,986, 5,980,905, 5,981,576, 5,985,902, 5,925,631, 5,990,148, 5,994,379, 5,994,381, 6,001,843, 6,002,014, 6,020,343, 6,025,353, 6,046,191, 6,071,936, 6,071,954, 6,077,869, 6,080,876, 6,083,969 and in WO 94/20480, WO 94/13635, WO 94/15932, WO 94/26731, WO 94/27980, WO 95/00501, WO 95/11883, WO 95/15315, WO 95/15316, WO 95/15318, WO 95/17317, WO 95/18799, WO 95/21817, WO 95/30652, WO 95/30656, WO 96/03392, WO 96/03385, WO 96/03387, WO 96/03388, WO 96/06840, WO 96/10021, WO 96/13483, WO 96/16934, WO 96/19469, WO 96/21667, WO 96/23786, WO 96/24584, WO 96/25405, WO 96/31509, WO 96/36623, WO 96/36617, WO 96/38418, WO 96/38442, WO 96/37467, WO 96/37468, WO 96/37469, WO 96/41626, WO 96/41645, WO 97/03953, WO 97/13767, WO 97/14691, WO 97/16435, WO 97/25045, WO 97/27181, WO 97/28120, WO 97/28121, WO 97/29776, WO 97/34882, WO 97/36863, WO 97/37984, WO 97/38986, WO 97/44027, WO 97/44028, WO 97/45420, WO 98/00416, WO 98/03484, WO 98/04527, WO 98/06708, WO 98/07714, WO 98/11080, WO 98/21195, WO 98/22442, WO 98/39330, WO 98/41511, WO 98/41516, WO 98/43649, WO 98/43966, WO 98/46594, WO 98/47509, WO 98/47871, WO 98/47890, WO 98/50033, WO 98/50075, WO 99/05104, WO 99/10331, WO 99/10332, WO 99/12930, WO 99/13799, WO 99/14194, WO 99/14195, WO 99/15205, WO 99/15503, WO 99/15505, WO 99/15513, WO 99/18960, WO 99/20110, WO 99/21585, WO 99/22720, WO 99/23087, WO 99/25695, WO 99/33796, WO 99/35130, WO 99/45913, WO 99/55830, WO 99/59634, WO 99/59635, WO 99/61016, WO 99/61436, WO 99/62884, WO 00/00200, WO 00/08024, WO 00/01380, WO 00/13685, WO 00/24719, WO 00/23433, WO 00/26216, WO 01/45703 and in EP 0 745 596 A1, EP 0 788 476 B1, EP 0 863 134 A1, EP 0 937 722 A1, and in co-pending U.S. application Ser. Nos. 10/024,046 and 10/102,865, and in co-pending Provisional Application Nos. 60/387,433, 60/391,769 and 60/392,044, the disclosures of each of which are incorporated by reference herein in their entirety.

The COX-2 inhibitors and their nitrosating and/or nitrosylated derivatives are disclosed in WO 01/45703 and in co-pending U.S. application Ser. Nos. 10/024,046 and 10/102,865, and in co-pending Provisional Application Nos. 60/387,433, 60/391,769 and 60/392,044, the disclosures of each of which are incorporated by reference herein in their entirety.

The methods for improving the gastroprotective properties of H₂ receptor antagonists, increasing the rate of ulcer healing, decreasing the rate of recurrence of ulcers, treating inflammations, treating ophthalmic diseases and disorders, treating microbial infections, decrease or reverse gastrointestinal toxicity and facilitate ulcer healing resulting from the administration of nonsteroidal antiinflammatory drugs (NSAIDs); improving the gastroprotective properties, anti-Helicobacter properties and antacid properties of H₂ receptor antagonists, preventing or treating gastrointestinal disorders, treating multiple sclerosis, and methods for treating viral infections, such as HIV disease, include those disclosed in WO 00/28988; the disclosure of which is incorporated by reference herein in its entirety. In these methods the organic nitrate compounds comprising at least one sulfur atom and/or at least one disulfide group can optionally be administered with at least one H₂ receptor antagonist that is optionally substituted with at least one NO and/or NO₂ group (i.e., nitrosylated and/or nitrosated).

Suitable H₂ receptor antagonists, include, but are not limited to, cimetidine, roxatidine, rantidine and the like. Suitable H₂ receptor antagonists are also described more fully in the literature, such as in Goodman and Gilman, The Pharmacological Basis of Therapeutics (9th Edition), McGraw-Hill, 1995, Pgs. 901-915; and the Merck Index on CD-ROM, Twelfth Edition, Version 12:1, 1996. The H₂ receptor antagonists and their nitrosating and/or nitrosylated derivatives are disclosed in WO 00/28988, the disclosure of which is incorporated by reference herein in its entirety.

The methods for treating gastrointestinal disorders, for improving the gastroprotective properties, anti-Helicobacter properties and antacid properties of proton pump inhibitors, for facilitating ulcer healing, for decreasing the rate of recurrence of ulcers, decrease or reverse gastrointestinal toxicity resulting from the administration of nonsteroidal antiinflammatory drugs (NSAIDs) and/or selective COX-2 inhibitors, for facilitate ulcer healing resulting from the administration of NSAIDs and/or selective COX-2 inhibitors, treating infections caused by Helicobacter pylori and/or viruses, include those disclosed in WO 00/50037, WO 01/66088 and WO 02/00166, the disclosure of each of which are incorporated by reference herein in their entirety. In these methods the organic nitrate compounds comprising at least one sulfur atom and/or at least one disulfide group can optionally be administered with at least one proton pump inhibitor that is optionally substituted with at least one NO and/or NO₂ group (i.e., nitrosylated and/or nitrosated), and/or at least one, at least one nitric oxide donor.

Suitable proton pump inhibitors, include, but are not limited to, omeprazole, lansoprazole, rabeprazole, pantoprazole, and the like. Suitable proton pump inhibitors are described more fully in the literature, such as in Goodman and Gilman, The Pharmacological Basis of Therapeutics (9th Edition), McGraw-Hill, 1995, Pgs. 901-915; and the Merck Index on CD-ROM, Twelfth Edition, Version 12:1, 1996. Proton pump inhibitors and their nitrosating and/or nitrosylated derivatives are also disclosed in disclosed in WO 00/50037, WO 00/61541, WO 99/45004, WO 01/12584, WO 01/66088, WO 00/61537 and WO 02/00166, the disclosures of each of which are incorporated by reference herein in their entirety.

The methods for treating and/or preventing sexual dysfunctions and/or enhancing sexual responses in patients, including males and females, include those disclosed in U.S. Pat. Nos. 5,932,538, 5,994,294, 5,874,437, 5,958,926 reissued as U.S. Pat. No. RE 03772346,172,060, 6,197,778, 6,177,428, 6,172,068, 6,221,881, 6,232,321, 6,197,782, 6,133,272, 6,211,179, 6,316,457 and 6,331,542, the disclosures of each of which are incorporated by reference herein in their entirety. In these methods the organic nitrate compounds comprising at least one sulfur atom and/or at least one disulfide group can optionally be administered with at least one vasoactive agent that is optionally substituted with at least one NO and/or NO₂ group (i.e., nitrosylated and/or nitrosated), and/or at least one, at least one nitric oxide donor.

Suitable vasoactive agents, include, but are not limited to those disclosed in U.S. Pat. Nos. 5,932,538, 5,994,294, 5,874,437, 5,958,926 reissued as U.S. Pat. No. RE 03772346,172,060, 6,197,778, 6,177,428, 6,172,068, 6,221,881, 6,232,321, 6,197,782, 6,133,272, 6,211,179, 6,316,457 and 6,331,542, the disclosures of each of which are incorporated by reference herein in their entirety. Vasoactive agents and their nitrosating and/or nitrosylated derivatives are also disclosed in U.S. Pat. Nos. 5,932,538, 5,994,294, 5,874,437, 5,958,926 reissued as U.S. Pat. No. RE 03772346,172,060, 6,197,778, 6,177,428, 6,172,068, 6,221,881, 6,232,321, 6,197,782, 6,133,272, 6,211,179, 6,316,457 and 6,331,542, the disclosures of each of which are incorporated by reference herein in their entirety.

The methods for prevent or treat diseases induced by the increased metabolism of cyclic guanosine 3′,5′-monophosphate (cGMP), such as hypertension, pulmonary hypertension, congestive heart failure, myocardial infraction, stable, unstable and variant (Prinzmetal) angina, atherosclerosis, cardiac edema, renal insufficiency, nephrotic edema, hepatic edema, stroke, asthma, bronchitis, chronic obstructive pulmonary disease (COPD), cystic fibrosis, dementia, immunodeficiency, premature labor, dysmenorrhoea, benign prostatic hyperplasis (BPH), bladder outlet obstruction, incontinence, conditions of reduced blood vessel patency, e.g., postpercutaneous transluminal coronary angioplasty (post-PTCA), peripheral vascular disease, allergic rhinitis, cystic fibrosis, and glucoma, and diseases characterized by disorders of gut motility, e.g., irritable bowel syndrome (IBS) include those disclosed in co-pending U.S. Pat. No. 6,331,542, the disclosure of which is incorporated by reference herein in its entirety. In these methods the organic nitrate compounds comprising at least one sulfur atom and/or at least one disulfide group can optionally be administered with at least one phosphodiesterase inhibitor that is optionally substituted with at least one NO and/or NO₂ group (i.e., nitrosylated and/or nitrosated).

Suitable phosphodiesterase inhibitors, include but are not limited to, filaminast, piclamilast, rolipram, Org 20241, MCI-154, roflumilast, toborinone, posicar, lixazinone, zaprinast, sildenafil, pyrazolopyrimidinones, motapizone, pimobendan, zardaverine, siguazodan, CI 930, EMD 53998, imazodan, saterinone, loprinone hydrochloride, 3-pyridinecarbonitrile derivatives, denbufyllene, albifylline, torbafylline, doxofylline, theophylline, pentoxofylline, nanterinone, cilostazol, cilostamide, MS 857, piroximone, milrinone, amrinone, tolafentrine, dipyridamole, papaverine, E4021, thienopyrimidine derivatives, triflusal, ICOS-351, tetrahydropiperazino(1,2-b)beta-carboline-1,4-dione derivatives, carboline derivatives, 2-pyrazolin-5-one derivatives, fused pyridazine derivatives, quinazoline derivatives, anthranilic acid derivatives, imidazoquinazoline derivatives, and in Goodman and Gilman, The Pharmacological Basis of Therapeutics (9th Ed.), McGraw-Hill, Inc. (1995), The Physician's Desk Reference (49th Ed.), Medical Economics (1995), Drug Facts and Comparisons (1993 Ed), Facts and Comparisons (1993), and The Merck Index (12th Ed.), Merck & Co., Inc. (1996), and the like. Phosphodiesterase inhibitors and their nitrosating and/or nitrosylated derivatives are also disclosed in U.S. Pat. Nos. 5,932,538, 5,994,294, 5,874,437, 5,958,926 reissued as U.S. Pat. No. RE 03772346,172,060, 6,197,778, 6,177,428, 6,172,068, 6,221,881, 6,232,321, 6,197,782, 6,133,272, 6,211,179, 6,316,457 and 6,331,542. The disclosures of each of which are incorporated herein by reference in their entirety.

The methods for preventing or treating benign prostatic hyperplasia, hypertension, congestive heart failure, variant (Printzmetal) angina, glaucoma, neurodegenerative disorders, vasospastic diseases, cognitive disorders, urge incontinence, or overactive bladder, or to reverse the state of anesthesia include those disclosed in co-pending U.S. application Ser. No. 09/387,724, allowed, assigned to NitroMed Inc., the disclosure of which is incorporated by reference herein in its entirety. In these methods the organic nitrate compounds comprising at least one sulfur atom and/or at least one disulfide group can optionally be administered with at least one α-adrenergic receptor antagonist that is optionally substituted with at least one NO and/or NO₂ group (i.e., nitrosylated and/or nitrosated), and/or at least one, at least one nitric oxide donor.

Suitable α-adrenergic receptor antagonist, include but are not limited to those disclosed in Goodman and Gilman, The Pharmacological Basis of Therapeutics (9th Ed.), McGraw-Hill, Inc. (1995), The Physician's Desk Reference (49th Ed.), Medical Economics (1995), Drug Facts and Comparisons (1993 Ed), Facts and Comparisons (1993), and The Merck Index (12th Ed.), Merck & Co., Inc. (1996), and in U.S. Pat. Nos. 5,932,538, 5,994,294, 6,294,517, 6,323,211, 6,417,162 and in co-pending U.S. application Ser. No. 09/387,724, allowed, assigned to NitroMed Inc. α-Adrenergic receptor antagonist and their nitrosating and/or nitrosylated derivatives are also disclosed in U.S. Pat. Nos. 5,932,538, 5,994,294, 6,294,517, 6,323,211, 6,417,162 and in co-pending U.S. application Ser. No. 09/387,724, allowed, assigned to NitroMed Inc., the disclosures of each of which are incorporated herein by reference in their entirety.

The methods for treating respiratory disorders, such as asthma, include those disclosed in U.S. Pat. No. 5,824,669, reissued as U.S. Pat. No. RE 037,611, 6,197,762, the disclosures of each of which are incorporated by reference herein in their entirety. In these methods the organic nitrate compounds comprising at least one sulfur atom and/or at least one disulfide group can optionally be administered with at least one steroid, β-agonist, anticholinergic, mast cell stabilizer or PDE inhibitor, that is optionally substituted with at least one NO and/or NO₂ group (i.e., nitrosylated and/or nitrosated).

Suitable steroids, β-agonists, anticholinergics, mast cell stabilizers and PDE inhibitors include those disclosed in U.S. Pat. No. 5,824,669, reissued as U.S. Pat. No. RE 037,611, 5,958,926 reissued as U.S. Pat. No. RE 0377234 and 6,197,762. The steroids, β-agonists, anticholinergics, mast cell stabilizers and PDE inhibitors and their nitrosating and/or nitrosylated derivatives are also disclosed in U.S. Pat. No. 5,824,669, reissued as U.S. Pat. No. RE 037,611, 5,958,926 reissued as U.S. Pat. No. RE 0377234 and 6,197,762, the disclosures of each of which are incorporated herein by reference in their entirety.

The methods for treating restenosis, include those disclosed in U.S. Pat. Nos. 6,087,479, 6,174,539, 6,255,277 and 6,352,709, and in WO 01/98286 and PCT/US00/04507, the disclosures of which are incorporated by reference herein in their entirety. In these methods the organic nitrate compounds comprising at least one sulfur atom and/or at least one disulfide group can optionally be administered with at least one steroid, taxane, rapamycin, tranilast, that is optionally substituted with at least one NO and/or NO₂ group (i.e., nitrosylated and/or nitrosated).

Suitable steroids and taxanes, including the nitrosating and/or nitrosylated derivatives, include those disclosed in U.S. Pat. No. 5,824,669, reissued as U.S. Pat. No. RE 037,611, and 6,197,762 and in WO 01/98286 and PCT/US00/04507, the disclosures of each of which are incorporated herein by reference in their entirety.

The organic nitrate compounds comprising at least one sulfur atom and/or at least one disulfide group, and, optionally, at least one steroid, taxane, rapamycin, tranilast, that is optionally substituted with at least one NO and/or NO₂ group (i.e., nitrosylated and/or nitrosated), can be incorporated into a natural or synthetic matrix which can then be applied with specificity to a biological site of interest. Accordingly the organic nitrate compound and the optionally substituted steroid, taxane, rapamycin, tranilast, and, optionally, NO donor is “bound to the matrix”. The incorporation of the compounds in to a matrix are disclosed in U.S. Pat. Nos. 6,087,479, 6,174,539, 6,255,277, 6,352,709, and in WO 01/98286 and PCT/US00/04507, the disclosures of each of which are incorporated herein by reference in their entirety.

When administered in vivo, the compounds and compositions of the invention can be administered in combination with pharmaceutically acceptable carriers and in dosages described herein. When the compounds and compositions of the invention are administered as a combination of at least one organic nitrate compounds comprising at least one sulfur atom and/or at least one disulfide group and/or therapeutic agent, they can also be used in combination with one or more additional compounds which are known to be effective against the specific disease state targeted for treatment. The organic nitrate compounds comprising at least one sulfur atom and/or at least one disulfide group, therapeutic agents and/or other additional compounds can be administered simultaneously with, subsequently to, or prior to administration of the organic nitrate compounds comprising at least one sulfur atom and/or at least one disulfide group.

The compounds and compositions of the invention can be administered by any available and effective delivery system including, but not limited to, orally, bucally, parenterally, by inhalation spray, by topical application, by injection, transdermally, or rectally (e.g., by the use of suppositories) in dosage unit formulations containing conventional nontoxic pharmaceutically acceptable carriers, adjuvants, and vehicles, as desired. Parenteral includes subcutaneous injections, intravenous, intramuscular, intrastemal injection, or infusion techniques.

Transdermal compound administration, which is known to one skilled in the art, involves the delivery of pharmaceutical compounds via percutaneous passage of the compound into the systemic circulation of the patient. Topical administration can also involve the use of transdermal administration such as transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions can include creams, sprays, lotions, gels, ointments, eye drops, nose drops, ear drops, and the like. In such dosage forms, the compositions of the invention can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions can contain polyethylene glycol 400. They can be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, can be impregnated with the compositions in solution, lotion, cream, ointment or other such form can also be used for topical application. The compositions can also be applied topically using a transdermal system, such as one of an acrylic-based polymer adhesive with a resinous crosslinking agent impregnated with the composition and laminated to an impermeable backing.

Solid dosage forms for oral administration can include capsules, tablets, effervescent tablets, chewable tablets, pills, powders, sachets, granules and gels. In such solid dosage forms, the active compounds can be admixed with at least one inert diluent such as sucrose, lactose or starch. Such dosage forms can also comprise, as in normal practice, additional substances other than inert diluents, e.g., lubricating agents such as magnesium stearate. In the case of capsules, tablets, effervescent tablets, and pills, the dosage forms can also comprise buffering agents. Soft gelatin capsules can be prepared to contain a mixture of the active compounds or compositions of the invention and vegetable oil. Hard gelatin capsules can contain granules of the active compound in combination with a solid, pulverulent carrier such as lactose, saccharose, sorbitol, mannitol, potato starch, corn starch, amylopectin, cellulose derivatives of gelatin. Tablets and pills can be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

Suppositories for vaginal or rectal administration of the compounds and compositions of the invention, such as for treating pediatric fever and the like, can be prepared by mixing the compounds or compositions with a suitable nonirritating excipient such as cocoa butter and polyethylene glycols which are solid at room temperature but liquid at rectal temperature, such that they will melt in the rectum and release the drug.

Injectable preparations, for example, sterile injectable aqueous or oleaginous suspensions can be formulated according to the known art using suitable dispersing agents, wetting agents and/or suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. Sterile fixed oils are also conventionally used as a solvent or suspending medium.

The compositions of this invention can further include conventional excipients, i.e., pharmaceutically acceptable organic or inorganic carrier substances suitable for parenteral application which do not deleteriously react with the active compounds. Suitable pharmaceutically acceptable carriers include, for example, water, salt solutions, alcohol, vegetable oils, polyethylene glycols, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran. Optionally, the suspension may also contain stabilizers.

The composition, if desired, can also contain minor amounts of wetting agents, emulsifying agents and/or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, and the like.

Various delivery systems are known and can be used to administer the compounds or compositions of the invention, including, for example, encapsulation in liposomes, microbubbles, emulsions, microparticles, microcapsules and the like. The required dosage can be administered as a single unit or in a sustained release form.

The bioavailabilty of the compositions can be enhanced by micronization of the formulations using conventional techniques such as grinding, milling, spray drying and the like in the presence of suitable excipients or agents such as phospholipids or surfactants.

The preferred methods of administration of the organic nitrate compounds comprising at least one sulfur atom and/or at least one disulfide group and compositions for the treatment of gastrointestinal disorders are orally, bucally or by inhalation. The preferred methods of administration for the treatment of inflammation and microbial infections are orally, bucally, topically, transdermally or by inhalation.

The compounds and compositions of the invention can be formulated as pharmaceutically acceptable salt forms. Pharmaceutically acceptable salts include, for example, alkali metal salts and addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids include, but are not limited to, hydrochloric, hydrobromic, hydroiodic, nitric, carbonic, sulfuric and phosphoric acid and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, β-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound.

While individual needs may vary, determination of optimal ranges for effective amounts of the compounds and/or compositions is within the skill of the art. Generally, the dosage required to provide an effective amount of the compounds and compositions, which can be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as the activity, efficacy, pharmacokinetic and toxicology profiles of the particular compound used, whether a drug delivery system is used, and whether the compound is administered as part of a drug combination.

The amount of a given organic nitrate compound comprising at least one sulfur atom and/or at least one disulfide group of the invention that will be effective in the treatment of a particular disorder or condition will depend on the nature of the disorder or condition, and can be determined by standard clinical techniques, including reference to Goodman and Gilman, supra; The Physician's Desk Reference, Medical Economics Company, Inc., Oradell, N.J., 1995; and Drug Facts and Comparisons, Inc., St. Louis, Mo., 1993. The precise dose to be used in the formulation will also depend on the route of administration, and the seriousness of the disease or disorder, and should be decided by the physician and the patient's circumstances.

The amount of the organic nitrate compound comprising at least one sulfur atom and/or at least one disulfide group in a pharmaceutical composition can contain about 1 to 300 mg of active compound per unit dosage form, such as a tablet or capsule. A liquid pharmaceutical composition can contain about 5 to 200 mg of active compound per liter. Pharmaceutical preparations containing a predetermined amount of one or several of the compounds according to this invention can be administered once daily in the form of slow or delayed release preparations, or several times a day at regular intervals, such as 2 to 3 times daily. About 5 to 300 mg, and desirably 20 to 300 mg, based on a patient body weight of 75 kg., of one or a combination of the effective agents can be administered per day to a patient. The compounds according to this invention can be administered in the form of injections 1 to 8 times daily or by means of an intravenous drip. Normally, an administration of about 5 to 200 mg/day are sufficient.

The invention also provides pharmaceutical kits comprising one or more containers filled with one or more of the ingredients of the pharmaceutical compounds and/or compositions of the invention, including, at least, one or more of the organic nitrate compounds comprising at least one sulfur atom and/or at least one disulfide group, and one or more of the NSAIDs, COX-2 inhibitors, H₂ receptor antagonists, proton pump inhibitor,s vasoactive agents, steroids, β-agonists, anticholinergics, mast cell stabilizers, PDE inhibitors, taxanes, rapamycins, tranilasts, that is optionally substituted with at least one NO and/or NO₂ group (i.e., nitrosylated and/or nitrosated), devices for administering the compositions, and notices in the form prescribed by a governmental agency regulating the manufacture, use or sale of pharmaceuticals or biological products which reflects approval by the agency of manufacture, use or sale for humans.

The disclosure of each patent, patent application and publication cited or described in the present specification is hereby incorporated by reference herein in its entirety.

Although the invention has been set forth in detail, one skilled in the art will appreciate that numerous changes and modifications can be made to the invention, and that such changes and modifications can be made without departing from the spirit and scope of the invention.

What is claimed is:

1. A method for treating, preventing and/or reducing inflammation, pain, and fever; for decreasing or reversing the gastrointestinal, renal and other toxicities resulting from the use of nonsteroidal antiinflammatory compounds; for treating and/or preventing gastrointestinal disorders; for facilitating wound healing; for treating inflammatory disease states and/or disorders; for treating and/or preventing ophthalmic diseases and/or disorders; for treating and/or improving gastrointestinal properties of COX-2 selective inhibitors; for treating and/or preventing renal toxicity; for treating and/or preventing COX-2 mediated disorders; for decreasing the recurrence of ulcers; for improving gastroprotective properties, anti-Helicobacter pylori properties or antacid properties of proton pump inhibitors; for treating and/or preventing bacterial infections, microbial infections, multiple sclerosis, and/or viral infections; for improving gastroprotective properties of H₂ receptor antagonists; for treating and/or preventing restenosis, autoimmune diseases, pathological conditions resulting from abnormal cell proliferation, polycystic kidney disease, inflammatory diseases or to inhibit wound contraction; for treating or preventing sexual dysfunctions in males and females, for enhancing sexual responses in males and females; for treating or preventing benign prostatic hyperplasia, hypertension, neurodegenerative disorders, vasospastic diseases, cognitive disorders, urge incontinence, or an overactive bladder; for reversing the state of anesthesia; for treating or preventing diseases induced by the increased metabolism of cyclic guanosine 3′,5′-monophosphate (cGMP) or for treating respiratory disorders, in a patient in need thereof comprising administering to the patient a therapeutically effective amount of at least one compound of Formula I or II, or a pharmaceutically acceptable salts thereof,
wherein the compound of Formula (I) is: 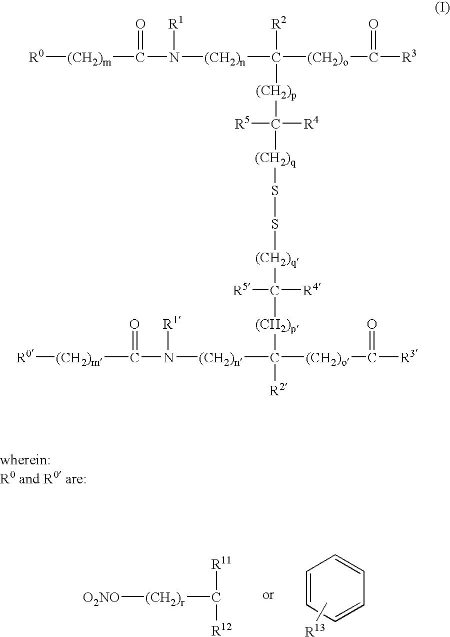
R¹¹ is hydrogen, an alkyl group having 1 to 6 carbon atoms, a substituted lower alkyl wherein the substituent is halogen, hydroxyl, lower alkoxy, aryloxy, amino, lower alkylamino, acylamino, acyloxy, arylamino, mercapto, lower alkylthio or arylthio,
R¹² is R¹¹ hydrogen or a lower alkyl group;
R¹³ is a nitratoalkyl group having 1 to 6 carbon atoms;
r is an integer from 0 to 10;
R¹ and R^{1′} are each independently hydrogen or lower alkyl;
R² and R^{2′} are each independently hydrogen, lower alkyl, phenyl, methoxyphenyl, phenyl-lower-alkyl, methoxyphenyl-lower-alkyl, hydroxyphenyl-lower-alkyl, hydroxy-lower-alkyl, alkoxy-lower-alkyl, amino-lower-alkyl, acylamino-lower-alkyl, mercapto-lower-alkyl or lower alkylthio-lower-alkyl;
R³ and R^{3′} are each independently hydroxyl, lower alkoxy, lower alkenoxy, di-lower-alkylamino-lower-alkoxy, acylamino-lower-alkoxy, acyloxy-lower-alkoxy, aryloxy, aryl-lower-alkoxy, substituted aryloxy or substituted aryl-lower-alkoxy, in which the substituent is methyl, halogen or methoxy; amino, lower alkylamino, di-lower-alkylamino, aryl-lower-alkylamino, hydroxy-lower-alkyl-amino, pyrrolidine, piperidine, morpholine, piperazine or amino-acid residues via peptide linkage;
R⁴ and R^{4′} are each independently hydrogen or lower alkyl;
R⁵ and R^{5′} are each independently R⁴, R^{4′} hydrogen or lower alkyl;
R² and R³, and R^{2′} and R^{3′}, can be linked together to form an ester or an amide;
R¹ and R², and R^{1′} and R^{2′}, can be linked together to form an alkylene bridge having 2 to 4 carbon atoms, an alkylene bridge having 2 to 3 carbon atoms and a sulfur atom, an alkylene bridge having 3 to 4 carbon atoms, which contains a double bond or an alkylene bridge, optionally substituted by hydroxyl, lower alkoxy, lower alkyl or di-lower-alkyl;
m, n, o, p, q, m′, n′, o′, p′ and q′ are each independently integers from 0 to 10;
wherein the compound of Formula (II) is: 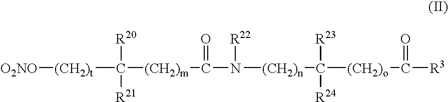
wherein:
R²⁰ and R²¹ are each independently a hydrogen, an alkyl having 1 to 6 carbon atoms, a substituted lower alkyl in which the substituent is a halogen, groups defined by R³ containing hydroxy, lower alkoxy, aryloxy, amino, lower alkylamino, acylamino, acyloxy, arylamino, mercapto, lower alkylthio or arylthio;
R²² is hydrogen or lower alkyl;
R²³ is hydrogen, lower alkyl, phenyl, methoxy phenyl, phenyl-lower alkyl, methoxyphenyl-lower alkyl, hydroxyphenyl-lower alkyl, hydroxy-lower alkyl, alkoxy-lower alkyl, amino-lower alkyl, acylamino-lower alkyl, mercapto-lower alkyl or lower alkylthio-lower alkyl;
R²⁴ is lower alkyl thiol, —SH, S-acyl compound of lower alkylthiol, preferably —S-acetyl, —S-propionyl, —S-butyryl, —S-isobutyryl, —S-capryl, —S-pivaloyl, —S-benzoyl; 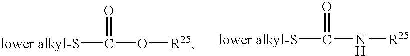
and lower alkylthio-lower alkanoic acid and esters and amides thereof, and lower alkylthio-lower alkyl;
R²⁵ is hydrogen and lower alkyl groups in which R³ and R²⁴ are bonded together and form part of a thiolactone group, groups in which R³ and R²³ are bonded together in the form of an ester or amide, groups in which R²² and R²³ are bonded together in the form of an alkylene bridge with 2 to 4 carbon atoms, an alkylene bridge with 2 to 3 carbon atoms and a sulfur atom, an alkylene bridge with 3 to 4 carbon atoms, which contains a double bond or an alkylene bridge as above, which can be substituted by one or more hydroxy, lower alkoxy, lower alkyl or di-lower alkyl groups; and
R³, m, n, and o are as defined herein.

2. The method of claim 1, further comprising administering a pharmaceutically acceptable carrier.

3. The method of claim 1, further comprising administering at least one NSAID, COX-2 inhibitor, H₂ receptor antagonist, proton pump inhibitor, vasoactive agent, steroid, β-agonist, anticholinergic, mast cell stabilizer, PDE inhibitor, taxane, rapamycin, tranilast, or mixture of two or more thereof.

4. The method of claim 1, wherein the compound of Formula (I) is:
N′-3-nitratopivaloyl-L-cysteinamide-glutathione mixed disulphide, N′-3-nitratopivaloyl-L-cysteine ethyl ester-glutathione mixed disulphide;
N′-3-nitratopivaloyl-L-cysteine ethyl ester-N′-acetyl-L-cysteine mixed disulphide;
N-(3-nitratopivaloyl)-L-cysteine ethyl ester-D,L-penicillamine mixed disulphide;
2-acetylamino-3-(2-(2,2-dimethyl-3-nitrooxy-propionylamino)-2-ethoxycarbon ylethyl disulphanyl)-3-methylbutyric acid;
N,N′-di(3-nitratopivaloyl)-L-cystine;
N,N′-di(3-nitratopivaloyl)-D,L-homocystine;
N,N′-di(3-nitratopivaloyl)-L-cystine diethyl ester;
N,N′-di(3-nitratopivaloyl)-D,L-homocystine diethyl ester;
N,N′-di(3-nitratopivaloyl)-L-cystine di-tertiary-butyl ester;
N,N′-di(4-nitratomethylbenzoyl)-L-cystine dimethyl ester;
N,N′-di(3-nitratomethylbenzoyl)-L-cystine dimethyl ester;
N,N′-di(4-nitratomethylbenzoyl)-L-cystine-di(N,N′-butylamide);
N,N′-di(3-nitratomethylbenzoyl)-L-cystine-di(N,N′-butylamide);
N,N′-di(4-nitratomethylbenzoyl)-L-cystinediamide;
N,N′-di(3-nitratomethylbenzoyl)-L-cystinediamide;
N,N′-di(3-nitratopivaloyl)-L-penicillamine disulphidediamide;
N,N′-di(3-nitratopivaloyl)-L-cystinediamide;
N,N′-di(3-nitratopivaloyl)-L-cystine-di(N,N′-methylamide);
N,N′-di(3-nitratopivaloyl)-L-cystine-di(N,N′-butylamide);
N,N′-di(3-nitratopivaloyl)-L-cystine-di(N,N′-tertiary-butylamide);
N,N′-di(3-nitratopivaloyl)-L-cystine-dimorpholide;
N,N′-di(3-nitratopivaloyl)-L-cystinediisopropyl ester, or a pharmaceutically acceptable salts thereof.

5. The method of claim 1, wherein the compound of Formula (II) is N-nitrato-pivaloyl-S-(N-acetyl-glycyl)-L-cysteine ethyl ester (compound SPM 5186);
N-nitrato-pivaloyl-S-(N-acetyl-alanyl)-L-cysteine ethyl ester (compound SPM 5185);
N-nitrato-pivaloyl-S-(N-acetyl-leucyl)-L-cysteine ethyl ester. N-(2-nitratoacetyl)-cysteine ethyl ester;
N-(2-nitratoacetyl)-S-acetyl-cysteine ethyl ester;
N-(2-nitratoacetyl)-S-propionyl-cysteine ethyl ester;
N-(2-nitratoacetyl)-S-pivaloyl-cysteine ethyl ester;
N-(2-nitratoacetyl)-methionine methyl ester;
N-(2-nitratopropionyl)-cysteine;
N-(2-nitratopropionyl)-cysteine ethyl ester;
N-(2-nitratopropionyl)-methionine ethyl ester;
N-(2-nitratobutyryl)-cysteine;
N-(2-nitratobutyryl)-cysteine ethyl ester;
N-(2-nitratobutyryl)-S-acetyl-cysteine ethyl ester;
N-(2-nitratobutyryl)-S-butyryl-cysteine ethyl ester;
N-(2-nitratobutyryl)-methionine ethyl ester;
N-(2-nitratoisobutyryl)-cysteine;
N-(2-nitratoisobutyryl)-cysteine ethyl ester;
N-(2-nitratoisobutyryl)-S-benzoyl-cysteine ethyl ester;
N-(2-nitratoisobutyryl)-S-acetyl-cysteine ethyl ester;
N-(2-nitratoisobutyryl)-S-pivaloyl-cysteine ethyl ester;
N-(2-nitratoisobutyryl)-methionine ethyl ester;
N-(3-nitratobutyryl)-cysteine;
N-(3-nitratobutyryl)-cysteine ethyl ester;
N-(3-nitratobutyryl)-S-acetyl-cysteine ethyl ester;
N-(3-nitratobutyryl)-S-propionyl-cysteine ethyl ester;
N-(3-nitratobutyryl)-methionine ethyl ester;
N-(3-nitratobutyryl)-homocysteine thiolactone;
N-(3-nitratopivaloyl)-cysteine;
N-(3-nitratopivaloyl)-cysteine ethyl ester;
N-(3-nitratopivaloyl)-cysteine ethyl ester-S-ethyl carbonate;
N-(3-nitratopivaloyl)-S-acetyl-cysteine ethyl ester;
N-(3-nitratopivaloyl)-S-propionyl-cysteine ethyl ester;
N-(3-nitratopivaloyl)-S-butyryl-cysteine ethyl ester;
N-(3-nitratopivaloyl)-S-isobutyryl-cysteine ethyl ester;
N-(3-nitratopivaloyl)-S-pivaloyl-cysteine ethyl ester;
N-(3-nitratopivaloyl)-S-benzoyl-cysteine ethyl ester;
N-(3-nitratopivaloyl)-methionine ethyl ester;
N-(3-nitratopivaloyl)-methionine;
N-(3-nitratopivaloyl)-homocysteine thiolactone;
N-(2-nitratohexanoyl)-cysteine ethyl ester;
N-(2-nitratohexanoyl)-S-propionyl-cysteine ethyl ester;
N-(3-nitratohexanoyl)-cysteine ethyl ester;
N-(3-nitratohexanoyl)-methionine methyl ester;
N-(12-nitratolauroyl)-cysteine;
N-(12-nitratolauroyl)-cysteine ethyl ester;
N-(12-nitratolauroyl)-S-acetyl-cysteine;
N-(12-nitratolauroyl)-S-pivaloyl-cysteine;
compound SPM 3672; compound SPM 6373; or a pharmaceutically acceptable salts thereof.
